(19) United States
(12) Patent Application Publication
(10) Pub. No.: US 2019/0388149 A1
(43) Pub. Date: Dec. 26, 2019

(54) OPTICAL ARRAY FOR TISSUE TREATMENT

(71) Applicant: Avava, Inc., Waltham, MA (US)

(72) Inventors: Charles Holland Dresser, Wayland, MA (US); Rajender Katkam, Boston, MA (US); Jayant Bhawalkar, Auburndale, MA (US)

(21) Appl. No.: 16/447,176

(22) Filed: Jun. 20, 2019

Related U.S. Application Data
(60) Provisional application No. 62/688,862, filed on Jun. 22, 2018.
(60) Provisional application No. 62/688,940, filed on Jun. 22, 2018.
(60) Provisional application No. 62/688,913, filed on Jun. 22, 2018.
(60) Provisional application No. 62/688,855, filed on Jun. 22, 2018.

(51) Int. Cl. A61B 18/20; A61B 5/00; A61N 5/06

(52) U.S. Cl. CPC A61B 18/203; A61B 18/201; A61B 2018/00458; A61N 5/0616; A61B 5/4836

(57) ABSTRACT

An optical system includes an array of optical elements configured to receive a primary laser beam and generate a plurality of sub-beams. The array of optical elements includes a plurality of optical elements that are configured to simultaneously focus the plurality of sub-beams to a plurality of focal regions in a target tissue. A pitch of the array of optical elements ranges from about 1 mm to about 3 mm. A numerical aperture of one or more optical elements of the plurality of optical element ranges from about 0.3 to about 1. A first sub-beam of the plurality sub-beams is configured to generate plasma in a first focal region of the plurality of focal regions.

### CROSS-REFERENCE TO RELATED APPLICATIONS

This application claims the benefit of U.S. Provisional Application No. 62/688,862, entitled “Multi-Lens Array For Tissue Treatment,” filed Jun. 22, 2018, U.S. Provisional Application No. 62/688,940, entitled “Pigment Detection for a Therapeutic Device,” filed Jun. 22, 2018, U.S. Provisional Application No. 62/688,913, entitled “Diffractive Optics For EMR-Based Tissue Treatment,” filed Jun. 22, 2018, and U.S. Provisional Application No. 62/688,855, entitled “Selective Plasma Generation for Tissue Treatment,” filed Jun. 22, 2018. The entirety of each of these applications is incorporated by reference.

### BACKGROUND

Melasma or chloasma faciei (the mask of pregnancy) is a common skin condition characterized by tan to dark gray-brown, irregular, well-demarcated macules and patches on the face. The macules are believed to be due to overproduction of melanin, which is taken up by the keratinocytes (epidermal melanosis) or deposited in the dermis (dermal melanosis, melanophages). The pigmented appearance of melasma can be aggravated by certain conditions such as pregnancy, sun exposure, certain medications (e.g., oral contraceptives), hormonal levels, and genetics. The condition can be classified as epidermal, dermal, or mixed depending on the location of excess melanin. Exemplary symptoms of melasma primarily include the dark, irregularly-shaped patches or macules, which are commonly found on the upper cheek, nose, upper lip, and forehead. These patches often develop gradually over time.

Unlike other pigmented structures that are typically present in the epidermal region of skin (e.g., at or near the tissue surface), dermal (or deep) melasma is often characterized by widespread presence of melanin and melanophages in portions of the underlying dermis. Accordingly, treatment of dermal melasma (e.g., lightening of the appearance of darkened pigmented regions) can be particularly challenging because of the greater difficulty in accessing and affecting such pigmented cells and structures located deeper within the skin. Accordingly, skin rejuvenation treatments such as facial peels (laser or chemical), dermabrasion, topical agents, and the like, which primarily affect the overlying epidermis often the first course of treatment for melasma, may not be effective in treating dermal melasma.

### SUMMARY

It has been observed that application of light or optical energy of certain wavelengths can be strongly absorbed by pigmented cells, thereby damaging them. However, an effective treatment of dermal melasma using optical energy introduces several obstacles. For example, pigmented cells in the dermis must be targeted with sufficient optical energy of appropriate wavelength(s) to disrupt or damage them. This damage or disruption may release or destroy some of the pigmentation and reduce the pigmented appearance. However, such energy can be absorbed by pigment (e.g., melanin) in the overlying skin tissue, such as the epidermis and upper dermis. This near-surface absorption can lead to excessive damage of the outer portion of the skin, and insufficient delivery of energy to the deeper dermis to affect the pigmented cells therein. Moreover, moderate thermal injury to melanin containing melanocytes located in the basal layer of the epidermis can trigger an increase in the production of melanin (e.g., hyperpigmentation) and severe thermal damage to the melanocytes can trigger a decrease in the production of melanin (e.g., hypopigmentation).

Approaches have been developed that involve application of optical energy to small, discrete treatment locations in the skin that are separated by healthy tissue to facilitate healing. Accurately targeting the treatment locations (e.g., located in dermal layer) with desirable specificity while avoiding damage to healthy tissue around the treatment location (e.g., in the epidermal layer) can be challenging. This requires, for example, an optical system with high numerical aperture (NA) for focusing a laser beam to a treatment location. The high NA optical system delivers a sufficiently high in-focus fluence (i.e., energy density) to the dermis, while maintaining a sufficiently low out-of-focus fluence in the epidermis (See U.S. Patent Application Publication No. 2016/0199132, entitled “Method and Apparatus for Treating Dermal Melasma”). This technique has been found to be advantageous for treatment of dermal pigmentation including Melasma in research settings.

However, this technique requires that a focal region having a small area (e.g., less than 0.002 cm²) is formed by the high NA optical system at a depth within a target tissue. Treatment is therefore only affected in a relatively small volume at the focal region. Melasma macules typically cover large areas (greater than 1 cm² or 500× larger than this small focal region) of a patient's skin. The area of tissue requiring treatment and the area of tissue at the focal region undergoing treatment are therefore different by orders of magnitude (e.g., 500×). For this reason, treatments employing this technique are relatively slow to complete (e.g., greater than a half an hour to treat 1 cm²) and require cumbersome movement of optical elements and a laser source. Treatments that require this much time are typically not widely adopted. This is because, they are labor intensive on the part of the clinician (e.g., doctor) and uncomfortable, tedious, and expensive on the part of the patient. In part for this reason, a laser-based system that effectively treats dermal pigmentation has yet to be made commercially available. Thus, patients currently suffering with dermal melasma are without an effective treatment for their condition.

As noted above, there is a present need for an optical system that allows for effective treatment of skin regions affected by undesired pigmented structures (e.g., dermal pigmentation) using a beam of electromagnetic radiation (EMR)—in a reasonable time duration (e.g., less than an hour). This can be achieved, for example, by treating multiple treatment locations simultaneously by incorporating a multi-lens array (or arrays of optical elements that generate quasi-diffraction free beams) in the optical system. The multi-lens array can receive a single EMR beam (e.g., a laser beam) having a large waist size that allows the laser beam to impinge on multiple lenses of the multi-lens array simultaneously. As a result, the input laser beam can be focused to multiple focal regions in the target tissue simultaneously.

In order to focus an EMR beam at a desired depth within a tissue (e.g., in the dermis of the skin tissue), it may be desirable for the multi-lens array to have a working distance greater than the desired depth. According to some embodiments, a window (e.g., window made of Sapphire) having a thickness ranging from about 0.5 mm to about 3 mm can be placed between the multi-lens array and the skin. The multi-lens array can have a working distance which is long enough to accommodate the window thickness as well as the desired depth of the focal region of the EMR beam into the skin. In order to have a working distance of a desirable length (e.g. between about 0.5 mm and about 5 mm) and a desirable NA, lens elements of the multi-lens array must have a diameter (or pitch) that is sufficiently large (e.g., greater than about 0.5 mm, between about 0.5 mm and about 5 mm, between about 1 mm and about 3 mm, etc.)

Additionally, current limitations of many commonly used lens array manufacturing processes do not allow for the manufacture of a multi-lens array that can receive a high power EMR beam and have the aforementioned properties (e.g., desirable working distance, desirable pitch, etc.)

Accordingly, improved methods, systems, and devices for EMR-based (e.g., laser-based) tissue treatment using multi-lens array are provided.

An optical system includes an array of optical elements configured to receive a primary laser beam and generate a plurality of sub-beams. The array of optical elements includes a plurality of optical elements that are configured to simultaneously focus the plurality of sub-beams to a plurality of focal regions in a target tissue. A pitch of the array of optical elements ranges from about 1 mm to about 3 mm. A numerical aperture of one or more optical elements of the plurality of optical element ranges from about 0.3 to about 1. A first sub-beam of the plurality sub-beams is configured to generate plasma in a first focal region of the plurality of focal regions.

In one implementation, the plurality of optical elements include a plurality of truncated lenses. In another implementation, the plurality of truncated lenses are arranged in at least one of a hexagonal array and a rectangular array.

In one implementation, a width of the plurality of optical elements ranges from about 1 mm to about 3 mm.

In one implementation, the optical system further includes a window configured to contact a tissue and transmit the plurality of sub-beams.

In one implementation, the first sub-beam is configured to generate plasma thermionically. In another implementation, the first sub-beam is configured to generate plasma optically.

In one implementation, the plurality of optical elements include a plurality of axicons. In yet another implementation, the first sub-beam is a quasi-diffraction-free-beam generated by a first axicon in the plurality of axicons.

In one implementation, the plurality of optical elements are held together by a holder configured to apply a lateral force on one or more optical elements of the plurality of optical elements.

A method includes receiving, by an array of optical elements comprising a plurality of optical elements, a primary laser beam. The method also includes generating, by the plurality of optical elements, a plurality of sub-beams focused at a plurality of focal regions in a target tissue. A pitch of the array of optical elements ranges from about 1 mm to about 3 mm. A numerical aperture of one or more optical elements of the plurality of optical element ranges from about 0.3 to about 1. A first sub-beam of the plurality sub-beams is configured to generate plasma in a first focal region of the plurality of focal regions.

In one implementation, the plurality of optical elements include a plurality of truncated lenses. In another implementation, the plurality of truncated lenses are arranged in at least one of a hexagonal array and a rectangular array.

In one implementation, a width of the plurality of optical elements ranges from about 1 mm to about 3 mm.

In one implementation, the plurality of optical elements include a plurality of axicons. In yet another implementation, the first sub-beam is a quasi-diffraction-free-beam generated by a first axicon in the plurality of axicons.

In one implementation, the method further includes contacting, using a window, a tissue and transmitting the plurality of sub-beams through the window.

In one implementation, the first sub-beam is configured to generate plasma thermionically. In another implementation, the first sub-beam is configured to generate plasma optically.

In one implementation, the plurality of optical elements are held together by a holder configured to apply a lateral force on one or more optical elements of the plurality of optical elements.

A tissue treatment system includes a laser system configured to emit a primary laser beam. The tissue treatment system also includes an array of optical elements configured to receive the primary laser beam and generate a plurality of sub-beams. The array of optical elements includes a plurality of optical elements which are configured to simultaneously focus the plurality of sub-beams to a plurality of focal regions in a target tissue. A pitch of the array of optical elements ranges from about 1 mm to about 3 mm. A numerical aperture of one or more optical elements of the plurality of optical element ranges from about 0.3 to about 1. A first sub-beam of the plurality sub-beams is configured to generate plasma in a first focal region of the plurality of focal regions.

### BRIEF DESCRIPTION OF THE DRAWINGS

Embodiments of the disclosure will be more fully understood from the following detailed description taken in conjunction with the accompanying drawings, in which:

FIG. 1 illustrates an exemplary embodiment of a treatment system;

FIG. 2 is a schematic illustration of a laser beam focused into a pigmented region of a dermal layer in skin;

FIG. 3A is an exemplary absorbance spectrum graph for melanin;

FIG. 3B is an exemplary absorbance spectrum graph for hemoglobin;

FIG. 4 illustrates a plot of the absorption coefficients of melanin and venous blood, and scattering coefficients of light in skin versus wavelength;

FIG. 5 illustrates an exemplary apparatus for tissue treatment;

FIG. 6A illustrates a side-view of an exemplary multi-lens array;

FIG. 6B illustrates a top-view of the multi-lens array of FIG. 6A;

FIG. 6C illustrates a top-view of another exemplary multi-lens array;

FIG. 7A illustrates a front view and a side-view of an exemplary aspheric lens;

FIG. 7B illustrates a front view and a side-view of an exemplary hexagonally truncated lens;

FIG. 7C illustrates an exemplary multi-lens array of hexagonally truncated lenses;

FIG. 8 illustrates an exemplary multi-lens array arranged on a mount;

FIG. 9 illustrates an optical element configured to generate a quasi-diffraction-free beam;

FIG. 10A illustrates a system comprising a multi-lens array and a window in contact with tissue, according to some embodiments;

FIG. 10B illustrates a single lenslet of a multi-lens array focusing a beamlet into a tissue, according to some embodiments;

FIG. 11 illustrates a multifocal multi-lens array, according to some embodiments;

FIG. 12A illustrates a variable focus lenslet assembly focusing to a first position, according to some embodiments;

FIG. 12B illustrates a variable focus lenslet assembly focusing to a second position, according to some embodiments; and,

FIG. 12C illustrates a variable focus lenslet assembly focusing to a third position, according to some embodiments.

It is noted that the drawings are not necessarily to scale. The drawings are intended to depict only typical aspects of the subject matter disclosed herein, and therefore should not be considered as limiting the scope of the disclosure. Those skilled in the art will understand that the systems, devices, and methods specifically described herein and illustrated in the accompanying drawings are non-limiting exemplary embodiments and that the scope of the present invention is defined solely by the claims.

### DETAILED DESCRIPTION

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

Embodiments of the disclosure are discussed in detail below with respect to treatment of pigmentary conditions of the skin, such as melasma, to improve the appearance of such a pigmentary condition. However, the disclosed embodiments can be employed for treatment of other pigmentary and non-pigmentary conditions and other tissue and non-tissue targets without limit. Examples of pigmentary conditions can include, but are not limited to, post inflammatory hyperpigmentation, dark skin surrounding eyes, dark eyes, café au lait patches, Becker's nevi, Nevus of Ota, congenital melanocytic nevi, freckles/lentigo, hemosiderin rich structures, pigmented gallstones, lutein, zeaxanthin, rhodopsin, carotenoid, biliverdin, bilirubin and hemoglobin rich structures, and tattoo-containing tissue. Examples of non-pigmentary conditions can include, but are not limited to, hair follicles, hair shaft, vascular lesions, infectious conditions, sebaceous glands, acne, and the like.

Further, in the present disclosure, like-named components of the embodiments generally have similar features, and thus within a particular embodiment each feature of each like-named component is not necessarily fully elaborated upon. Additionally, to the extent that linear or circular dimensions are used in the description of the disclosed systems, devices, and methods, such dimensions are not intended to limit the types of shapes that can be used in conjunction with such systems, devices, and methods. A person skilled in the art will recognize that an equivalent to such linear and circular dimensions can easily be determined for any geometric shape. Sizes and shapes of the systems and devices, and the components thereof, can depend at least on the anatomy of the subject in which the systems and devices will be used, the size and shape of components with which the systems and devices will be used, and the methods and procedures in which the systems and devices will be used.

In general, high numerical aperture (NA) optical treatment systems are described that can focus electromagnetic radiation (EMR) (e.g., a laser beam) to a treatment region in a tissue. The focused laser beam can deliver optical energy to the treatment region without harming the surrounding tissue. The delivered optical energy can, for example, disrupt pigmented chromophores and/or targets in a treatment region of the dermal layer of the skin, without affecting the surrounding regions (e.g., overlying epidermal layer, other portions of the dermal layer, and the like). The delivered optical energy can also disrupt pigmented target areas of the skin or tissue surrounded by unaffected/non-target regions. In other implementations, the delivered optical energy can cause tattoo removal, alteration, or hemoglobin-related treatment.

Exemplary methods and devices for treating skin conditions with light or optical energy are disclosed in U.S. Patent Application Publication No. 2016/0199132, entitled “Method and Apparatus for Treating Dermal Melasma,” and U.S. Provisional Application No. 62/438,818, entitled “Method and Apparatus for Selective Treatment of Dermal Melasma,” each of which is incorporated by reference herein in their entirety.

In general, systems and corresponding methods are provided for treatment of pigmentary conditions in tissues. As discussed in greater detail below, the disclosed systems and methods employ electromagnetic radiation (EMR), such as laser beams, to deliver predetermined amounts of energy to a target tissue. The EMR can be focused to a focal region and the focal region can be translated or rotated in any direction with respect to the target tissue. The predetermined amount of radiation can be configured to thermally disrupt or otherwise damage portions of the tissue exhibiting the pigmentary condition. In this manner, the predetermined amount of energy can be delivered to any position within the target tissue for treatment of the pigmentary condition such as to improve the appearance thereof.

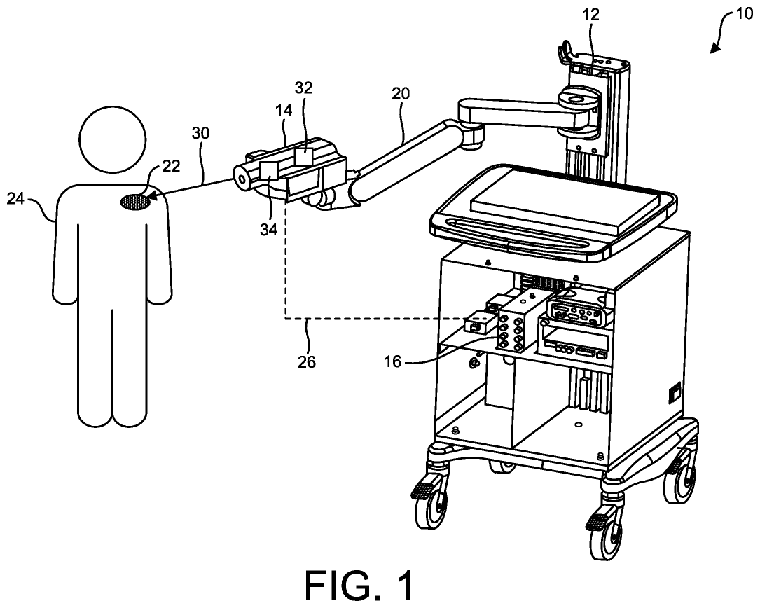

FIG. 1 illustrates one exemplary embodiment of a treatment system 10. As shown, the treatment system 10 includes a mounting platform 12, an emitter 14, and a controller 16. The mounting platform 12 can include one or more manipulators or arms 20. The arms 20 can be coupled to the emitter 14 for performing various treatments on a target tissue 22 of a subject 24. Operation of the mounting platform 12 and emitter 14 can be directed by a user, manually or by using the controller 16 (e.g., via a user interface). In certain embodiments (not shown), the emitter can have a hand-held form factor and the mounting platform can be omitted. In other embodiments, the mounting platform can be a robotic platform and the arms can be communicatively coupled to the controller for manipulation of the emitter.

The emitter 14 and controller 16 (and optionally the mounting platform 12) can be in communication with one another via a communications link 26, which can be any suitable type of wired and/or wireless communications link carrying any suitable type of signal (e.g., electrical, optical, infrared, etc.) according to any suitable communications protocol.

Embodiments of the controller 16 can be configured to control operation of the emitter 14. In one aspect, the controller 16 can control movement of EMR 30. As discussed in detail below, the emitter 14 can include a source 32 for emission of the EMR 30 and a scanning system 34 for manipulation of the EMR 30. As an example, the scanning system 34 can be configured to focus EMR 30 to a focal region and translate and/or rotate this focal region in space. The controller 16 can send signals to the source 32, via the communications link 26 to command the source 32 to emit the EMR 30 having one or more selected properties, such as wavelength, power, repetition rate, pulse duration, pulse energy, focusing properties (e.g., focal volume, Raleigh length, etc.). In another aspect, the controller 16 can send signals to the scanning system 34, via the communications link 26 to command the scanning system 34 to move the focal region of the EMR 30 with respect the target tissue 22 in one or more translation and/or rotation operations.

Embodiments of the treatment system 10 and methods are discussed herein in the context of targets within skin tissue, such as a dermal layer. However, the disclosed embodiments can be employed for treatment of any tissue in any location of a subject, without limit. Examples of non-skin tissues can include, but are not limited to, surface and sub-surface regions of mucosal tissues, genital tissues, internal organ tissues, and gastrointestinal tract tissues.

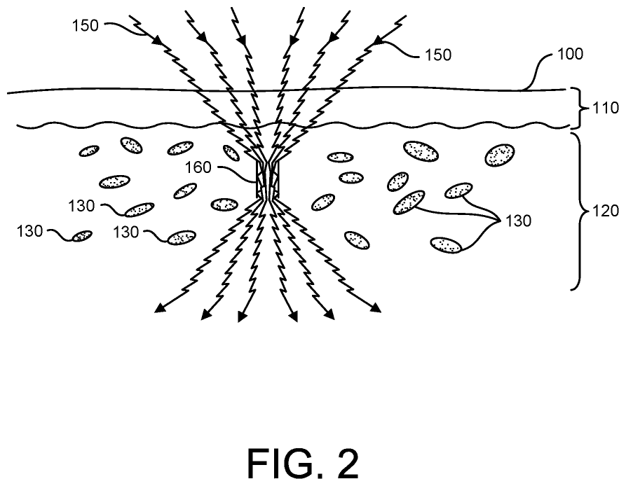

FIG. 2 is a schematic view of an illustration of a laser beam focused into a pigmented region of a dermal layer in a skin tissue. The skin tissue includes a skin surface 100 and an upper epidermal layer 110, or epidermis, which can be, e.g., about 60-120 μm thick in the facial region. The epidermis 110 can be slightly thicker in other parts of the body. For example, in general the thickness of the epidermis can range from about 30 μm (e.g., on the eyelids) to about 1500 μm (e.g., on the palm of the hand or soles of the feet). Such epidermis may be thinner or thicker than the examples above in certain conditions of the skin, for example psoriasis. The underlying dermal layer 120, or dermis, extends from below the epidermis 110 to the deeper subcutaneous fat layer (not shown). Skin exhibiting deep or dermal melasma can include a population of pigmented cells or regions 130 that contain excessive amounts of melanin. Electromagnetic radiation (EMR) 150 (e.g., a laser beam) can be focused into one or more focal regions 160 that can be located within the dermis 120, or the epidermis, 110. The EMR 150 can be provided at one or more appropriate wavelengths that can be absorbed by melanin. EMR wavelength(s) can be selected based on one or more criteria described below.

### Properties of Treatment Radiation

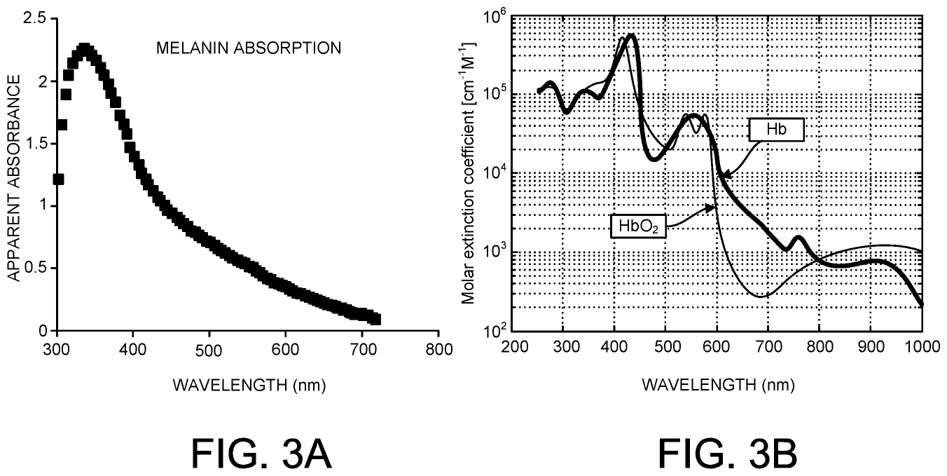

Determination of desirable wavelength for treatment of certain skin conditions, such as pigmentary conditions and non-pigmentary conditions, can depend on, for example, the wavelength dependent absorption coefficient of the various competing chromophores (e.g., chromophore, hemoglobin, tattoo ink, etc.) present in the skin. FIG. 3A is an exemplary absorbance spectrum graph for melanin. The absorption of EMR by melanin is observed to reach a peak value at a wavelength of about 350 nm, and then decreases with increasing wavelength. Although absorption of the EMR by the melanin facilitates heating and/or disruption of the melanin-containing regions 130, a very high melanin absorbance can result in high absorption by pigment in the epidermis 110 and reduced penetration of the EMR into the dermis 120, or the epidermis 110. As illustrated in FIG. 3A, melanin absorption is relatively high at EMR wavelengths that are less than about 500 nm. Accordingly, wavelengths less than about 500 nm may not be suitable for penetrating sufficiently into the dermis 120 to heat and damage or disrupt pigmented regions 130 therein. Such enhanced absorption at smaller wavelengths can result in unwanted damage to the epidermis 110 and upper (superficial) portion of the dermis 120, with relatively little unabsorbed EMR passing through the tissue into the deeper portions of the dermis 120.

FIG. 3B is an exemplary absorbance spectrum graph for oxygenated or deoxygenated hemoglobin. Hemoglobin is present in blood vessels of skin tissue and can be oxygenated (HbO₂) or deoxygenated (Hb). Each form of Hemoglobin may exhibit slightly different EMR absorption properties. As illustrated in FIG. 3B, exemplary absorption spectra for both Hb and HbO₂ indicate a high absorption coefficient for both Hb and HbO₂ at EMR wavelengths less than about 600 nm, with the absorbance decreasing significantly at higher wavelengths. Strong absorption of EMR directed into skin tissue by hemoglobin (Hb and/or HbO₂) can result in heating of the hemoglobin-containing blood vessels, resulting in unwanted damage to these vascular structures and less EMR available to be absorbed by the melanin.

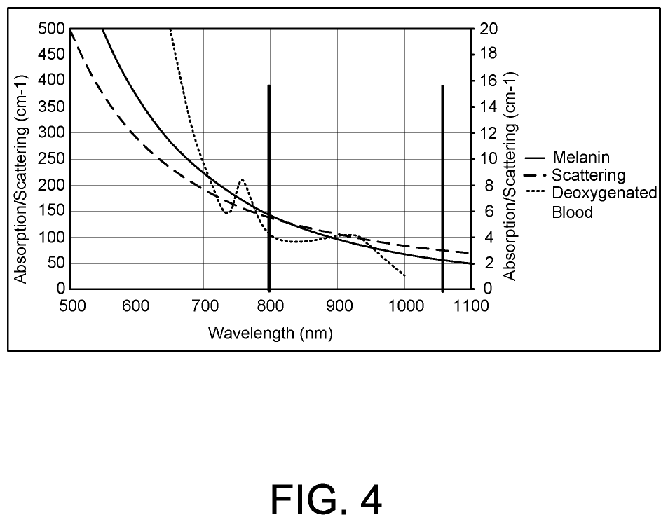

The choice of an appropriate wavelength for EMR can also depend on wavelength dependent scattering profile of tissues interacting with the EMR. FIG. 4 illustrates a plot of the absorption coefficient of melanin and venous (deoxygenated) blood versus wavelength. FIG. 4 also illustrates a plot of the scattering coefficient of light in skin versus wavelength. Absorption in melanin decreases monotonically with wavelength. If melanin is the target of a pigmentary condition treatment, a wavelength having a high absorption in melanin is desirable. This would suggest that the shorter the wavelength of light, the more efficient the treatment. However, absorption by blood increases at wavelengths shorter than 800 nm, thereby increasing the risk of unintentional targeting of blood vessels. In addition, as the intended target can be located below the skin surface, the role of scattering by skin (e.g., dermal layer) can be significant. Scattering reduces the amount of light that reaches the intended target. The scattering coefficient decreases monotonically with increasing wavelength. Therefore, while a shorter wavelength can favor absorption by melanin, a longer wavelength can favor deeper penetration due to reduced scattering. Similarly, longer wavelengths are better for sparing blood vessels due to a lower absorption by blood at longer wavelengths.

With the above considerations in mind, wavelengths can range from about 400 nm to about 4000 nm, and more particularly about 500 nm to about 2500 nm, can be used for targeting certain structures (e.g., melanin) in the dermis. In particular, wavelengths of about 800 nm and about 1064 nm can be useful for such treatments. The 800 nm wavelength can be attractive because laser diodes at this wavelength are less costly and readily available. However, 1064 nm can be particularly useful for targeting deeper lesions due to lower scattering at this wavelength. A wavelength of 1064 nm can also be more suitable for darker skin types in whom there is a large amount of epidermal melanin. In such individuals the higher absorption of lower wavelength EMR (e.g., about 800 nm) by melanin in the epidermis increases the chances of thermal injury to the skin. Hence, 1064 nm may be a more suitable wavelength of the treatment radiation for certain treatments for some individuals.

Various laser sources can be used for the generation of EMR. For example, Neodymium (Nd) containing laser sources are readily available that provide 1064 nm EMR. These laser sources can operate in a pulsed mode with repetition rates in a range of about 1 Hz to 100 kHz. Q-Switched Nd lasers sources may provide laser pulses having a pulse duration of less than one nanosecond. Other Nd laser sources may provide pulses having pulse durations more than one millisecond. An exemplary laser source providing 1060 nm wavelength EMR is a 20 W NuQ fiber laser from Nufern of East Granby, Conn., USA. The 20 W NuQ fiber laser provides pulses having a pulse duration of about 100 ns at a repetition rate in the range between about 20 kHz and about 100 kHz. Another laser source, is an Nd:YAG Q-smart 850 from Quantel of Les Ulis, France. The Q-smart 850 provides pulses having a pulse energy up to about 850 mJ and a pulse duration of about 6 ns at a repetition rate of up to about 10 Hz.

The systems described herein can be configured to focus the EMR in a highly convergent beam. For example, the system can include a focusing or converging lens arrangement having a numerical aperture (NA) selected from about 0.3 to 1.0 (e.g., between about 0.5 and about 0.9). The correspondingly large convergence angle of the EMR can provide a high fluence and intensity in the focal region of the lens (which can be located within the dermis) with a lower fluence in the overlying tissue above the focal region. Such focal geometry can help reduce unwanted heating and thermal damage in the overlying tissue above the pigmented dermal regions. The exemplary optical arrangement can further include a collimating lens arrangement configured to direct EMR from the emitting arrangement onto the focusing lens arrangement.

The exemplary optical treatment systems can be configured to focus the EMR to a focal region having a width or spot size that is less than about 500 μm, for example, less than about 200 μm less than about 100 μm, or even less than about 50 μm) e.g., as small as about 1 μm). For example, the spot size can range from about 1 μm to about 50 μm, from about 50 μm to about 100 μm, and from about 100 μm to about 500 μm. The spot size of the focal region can be determined, for example, in air. Such spot size can be selected as a balance between being small enough to provide a high fluence or intensity of EMR in the focal region (to effectively irradiate pigmented structures in the dermis), and being large enough to facilitate irradiation of large regions/volumes of the skin tissue in a reasonable treatment time. The exemplary optical arrangement can also be configured to direct the focal region of the EMR onto a location within the dermal tissue that is at a depth below the skin surface, such as in the range from about 120 μm to about 1000 μm, e.g., between about 150 μm to about 500 μm.

Such exemplary depth ranges can correspond to typical observed depths of pigmented regions in skin that exhibits dermal melasma or other targets of interest. This focal depth can correspond to a distance from a lower surface of the apparatus configured to contact the skin surface and the location of the focal region. Additionally, some embodiments can be configured for treating targets within the epidermis. For example, an optical arrangement may be configured to direct a focal region of the EMR to a location within the epidermis tissue, for example in a range from about 5 μm to 2000 μm beneath the skin surface. Still other embodiments may be configured for treating a target deep in the dermis. For example, a tattoo artist typically calibrates his tattoo gun to penetrate the skin to a depth from about 1 mm to about 2 mm beneath the skin surface. Accordingly, in some embodiments an optical arrangement may be configured to direct a focal region of the EMR to a location within the dermis tissue in a range from about 0.4 mm to 2 mm beneath the skin surface.

A large treatment region (e.g., several square centimeters) of a target tissue can be treated by scanning an EMR (e.g., laser beam) over the treatment region. For example, an optical system emitting an EMR can traverse over the treatment region such that the EMR impinges on multiple locations in the treatment region. Examples of scanning include: tipping/tilting an array of focal regions, rotating the array of focal regions, and translating the array of focal regions. Further description of relevant scanning means is described in U.S. patent application Ser. No. 16/219,809 “Electromagnetic Radiation Beam Scanning System and Method,” to Dresser et al., incorporated herein by reference. Alternately, the optical system can remain fixed with respect to the treatment region and can vary the direction of the emitted EMR such that the EMR scans over the treatment region. However, these scanning techniques can be time consuming, and therefore may not be desirable (e.g., when the treatment region is large). Time taken to treat a treatment region can be reduced by using a laser beam having a large cross-section (e.g., in a range between about 3 mm and about 30 mm) and simultaneously generating multiple sub-beams using a multi-lens array. The various sub-beams can simultaneously treat multiple locations of the treatment region.

The lenses of the multi-lens array can have a large NA (e.g., ranging from about 0.3 to about 1), and can focus the various sub-beams to multiple focal regions in the treatment region of a target tissue (e.g., dermis in a skin tissue). The sub-beams can generate plasma in the focal regions without adversely affecting the overlying layers of the target tissue (e.g., epidermis of the skin tissue). In some embodiments, plasma may be generated selectively through thermionic plasma generation. In alternative embodiments, the plasma may be generated through optical breakdown. The width of the lenses of the multi-lens array can range from about 1 mm to about 3 mm. The lenses in the multi-lens array can be designed to reduce inter-lens spacing. For example, the lenses can be generated by truncating an aspherical lens (e.g., truncating the aspherical lens into a polygon shape). The truncated lenses can be arranged abutting one another along their respective edges (e.g., arranged on a mount). The above-mentioned ranges of NA and/or width of the lenses and/or truncated shape of the lenses in the multi-lens array can allow for efficient treatment of underlying layers of the target tissue (e.g., dermis in skin tissue) without undesirable effect on the overlying layers of the target tissue (e.g., epidermis in skin tissue).

Commonly used lens arrays (e.g. microlens arrays) can include a thin-film coating, and can be manufactured using manufacturing processes such as lithography, micro/nano-molding, ion-beam milling, and the like. These manufacturing processes do not allow for production of a large sagittal height (sag). Therefore, these manufacturing processes may not allow for production of multi-lens arrays having large numerical apertures (e.g. greater than 0.3, between about 0.3 and about 1) and large pitches (e.g. greater than 1 mm, between about 1 mm and about 3 mm, etc.) For example, a plano-convex lens element having an index of refraction of about 1.5, a width of about 3 mm, and a focal length of about 3 mm, can have a radius of curvature of about 1.5 mm and a change in thickness over the size of the lens of about 1.5 mm. Micro-lens array manufacturing methods described above can only accommodate a small change in thickness (e.g. sagittal height (sag)) of about 60 microns. Therefore, common lens array manufacturing methods may not lend themselves to production of the multi-lens arrays described in this application.

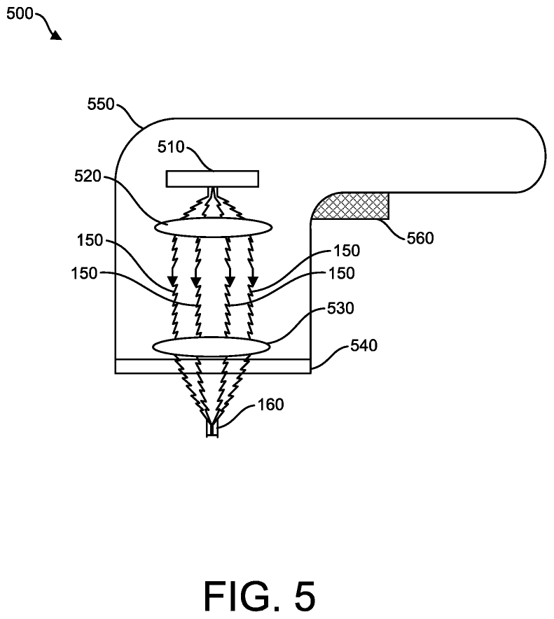

FIG. 5 illustrates an exemplary apparatus 500 for tissue treatment (e.g., treatment of dermal melasma) using EMR 150 (e.g., a laser beam). For example, the apparatus 500 can include a radiation emitter arrangement 510 (e.g., a laser system), and an optical arrangement that can be provided between the radiation emitter arrangement 510 and the target tissue to be treated. For example, the optical arrangement can include a first lens arrangement 520 and a second lens arrangement 530. These exemplary components can optionally be provided in a handpiece 550 or other housing or enclosure. The apparatus 500 can further include a plate 540 having a lower surface configured to contact a surface of the target tissue being treated. An actuator arrangement 560 can be provided to control the operation of the apparatus 500 (e.g., to activate and/or turn off the emitter arrangement 510, control or adjust certain operational parameters of the apparatus 500, etc.). A power source (not shown) for the radiation emitter arrangement 510 can be provided. For example, the power source can include a battery provided within the handpiece 550, an electrical cord or other conductive connection provided between the emitter arrangement 510 and an external power source (e.g. an electrical outlet or the like), etc.

The radiation emitter arrangement 510 can include, for example, one or more laser diodes, optical fibers, waveguides, or other components configured to generate and/or emit EMR 150, and direct it toward or onto the optical arrangement (e.g., onto the first lens arrangement 520). In certain exemplary embodiments of the present disclosure, the radiation emitter arrangement 510 can include one or more laser diodes that emit optical radiation 150 having one or more wavelengths between about 400 nm and about 1100 nm (e.g., between about 650 nm and about 750 nm).

In further exemplary embodiments of the present disclosure, the radiation emitter arrangement 510 can include distal ends of one or more waveguides (e.g., optical fibers not shown). The waveguides can be configured or adapted to direct EMR 150 from an external source (not shown) toward or onto the first lens arrangement 520. Such exemplary external EMR source can be configured to provide or direct EMR 150 to the radiation emitter arrangement 510 having one or more wavelengths between about 400 nm and about 1100 nm (e.g., between about 650 nm and about 750 nm).

In further exemplary embodiments of the present disclosure, the electromagnetic radiation (EMR) 150 can be focused into one or more focal regions 160 that can be located within the target tissue (e.g., within dermis 120). The second lens arrangement 530 can serve as a focusing lens that includes, for example, a single objective lens as shown in FIG. 5, an array of plano-convex lenses or cylindrical lenses, axicons, or the like. As described below, the second lens arrangement 530 can be a multi-lens array that includes multiple lenses. The lenses of the multi-lens array can each have high NA (e.g., between about 0.3 and about 1). The lenses of the multi-lens array can receive the EMR 150 and can generate multiple sub-beams focused at multiple focal regions in the target tissue. The focal regions can have a high local intensity of EMR (e.g., about 10⁵ W/cm² to about 10¹⁵ W/cm²). Plasma generation in the target tissue can be localized in the focal region. For example, if the focal region is located in the dermis, plasma can be generated in the dermis without affecting the overlying epidermis.

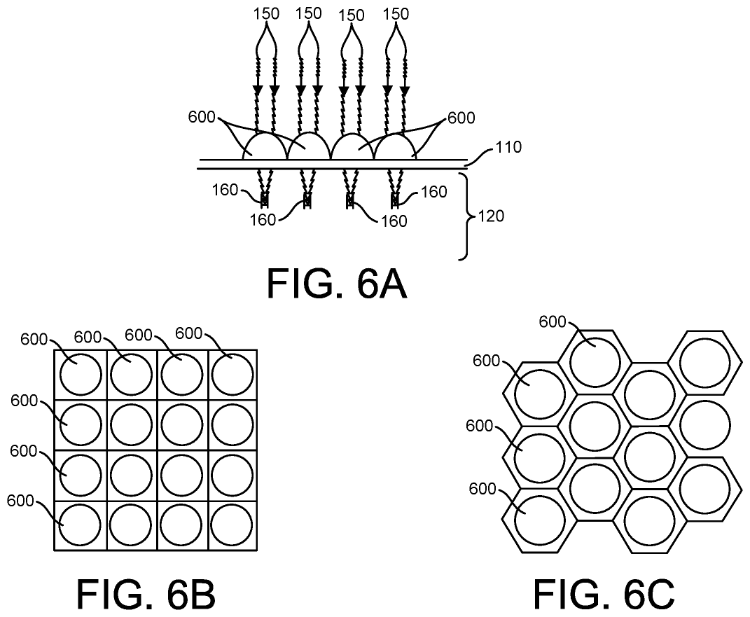

In further exemplary embodiments of the present disclosure, the second lens arrangement 530 can include an array of lenses 600, e.g., as provided in a schematic side view of the exemplary configuration illustrated in FIG. 6A. For example, the lenses 600 can include any conventional type of convergent lenses (e.g., convex lenses or plano-convex lenses) and/or optical elements for generating quasi-diffraction-free-beam (e.g., axicons). The lenses 600 can be configured to focus EMR 150 into a plurality of focal regions 160 within the underlying dermis 120, as illustrated in FIG. 6A.

Each of the lenses can have a large NA (e.g., between about 0.3 and 1), such that the EMR 150 converges from a relatively wide area at or near a tissue surface (with a relatively low intensity or local fluence) to a small width (with higher intensity or local fluence) in the focal region 160 within the tissue (e.g., within the dermis 120). Such optical properties can provide a sufficient intensity of EMR 150 within the focal region 160 to damage pigmented cells that absorb the radiation 150, while avoiding areas or volumes of high fluence or intensity away from the volume of dermis 120 containing pigmented cells 130, thereby reducing likelihood of damaging overlying, underlying, and/or adjacent volumes of unpigmented target tissue.

The lenses 600 can be provided in a substantially square or rectangular array, such as that shown in the top view of such exemplary configuration in FIG. 6B. According to further exemplary embodiments of the present disclosure, the lenses 600 can be provided in a hexagonal array as shown in FIG. 6C. Other exemplary patterns and/or shapes of the lenses 600 can be provided in still further exemplary embodiments. A width of the lenses 600 can range from about 1 mm to about 5 mm. The exemplary lenses 600 that are slightly wider or narrower than this can also be provided in certain exemplary embodiments.

In additional exemplary embodiments of the present disclosure, the radiation emitter arrangement 510 and/or the first lens arrangement 520 can be configured to direct a single wide beam of EMR 150 (such as, e.g., that shown in FIG. 5) over the entire array of lenses 600 or a substantial portion thereof. Such exemplary configuration can generate a plurality of focal regions 160 in the dermis 120 simultaneously. In further exemplary embodiments, the radiation emitter arrangement 510 and/or the first lens arrangement 520 can be configured to direct a plurality of smaller beams of EMR 150 onto individual ones of the lenses 600. According to still further exemplary embodiments, the radiation emitter arrangement 510 and/or the first lens arrangement 520 can be configured to direct one or more smaller beams of EMR 150 onto a portion of the array of lenses 600, e.g. onto a single micro-lens or a plurality of the lenses 600, and the smaller beam(s) can be scanned over the array of the lenses 600, such that a plurality of the focal regions 160 can be generated sequentially or non-simultaneously in the dermis 120.

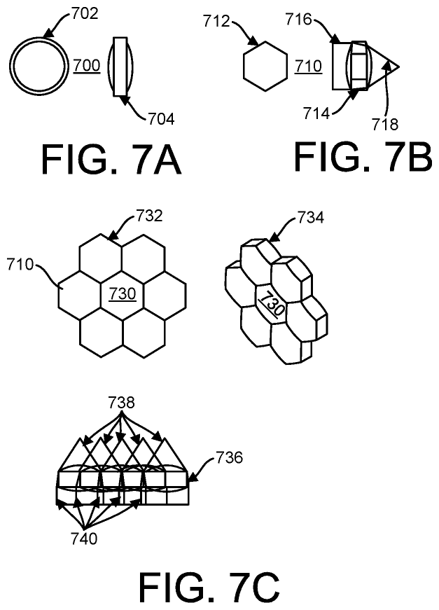

An exemplary multi-lens array and some of its components are shown according to some embodiments in FIGS. 7A-7C. FIG. 7A illustrates a front view 702 and a side-view 704 of an exemplary aspheric lens 700 (e.g., Thorlabs PN 355390-C). The aspheric lens can have an NA ranging from about 0.3 to about 1 (e.g., NA of about 0.55), and an effective focal length within the range from about 1 mm to about 3 mm (e.g., about 2.75 mm). According to some embodiments, a conventional aspheric lens 700 can be modified to create a multi-lens array.

FIG. 7B illustrates a front view 712 and a side-view 714 of an exemplary truncated lens 710. The truncated lens 710 can be obtained, for example, by truncating the aspheric lens 700 (e.g., truncating in a hexagonal pattern). According to some embodiments, truncation can be performed by at least one diamond turning and conventional lens polishing and grinding techniques. According, to some embodiments, the truncated lens 710 can be manufactured directly without having to be truncated from an aspheric lens 700. As shown in the side view 714, the truncated lens 714 can receive a collimated light beam 716 and emit a focused light beam 718.

FIG. 7C illustrates an exemplary multi-lens array 730 of hexagonally truncated lenses 710. The multi-lens array 730 can include several truncated lenses 710 that can be adhered to one another (e.g., using adhesives) along their edges. The truncated lenses 710 can be arranged, for example, in a hexagonal array, a rectangular array, and the like. According to some embodiments, it can be desirable that the truncated lenses 710 are not adhered to one another with an adhesive (e.g., when the multi-lens array 730 is exposed to high peak power applications). The multi-lens array 730 is shown in a front view 732, an isometric view 734, and a side view 736. As illustrated in the side view 736, the multi-lens array 730 can receive an input laser beam 740 that that can impinge on several lenses of the multi-lens array 730. The lenses of the multi-lens array 730 can focus portions of the input laser beam 740 (e.g., collimated laser beam) to multiple focused sub-beams (e.g., seven focused light beams 738).

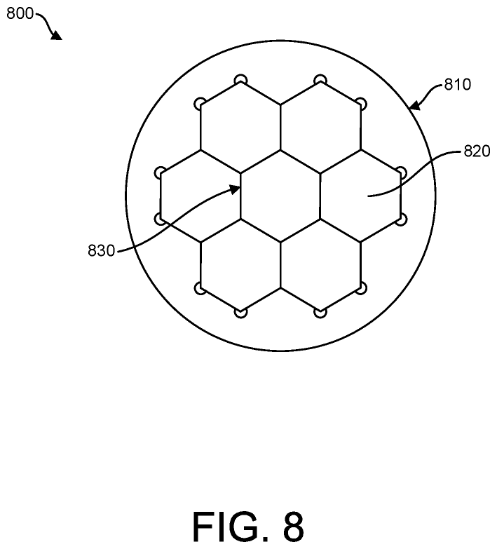

FIG. 8 illustrates an exemplary multi-lens array 800 arranged on a mount. The mount 810 can hold multiple lenses 820 (e.g., hexagonally truncated lens 710). In some embodiments, the mount 810 can provide a lateral force (e.g., compressive forces in the plane of the lens) on the multi-lens array 800 to hold the multiple lenses 820 together. This can allow the multiple lenses 820 to be affixed to one another without an adhesive in the inter-lens region 830. This can be advantageous, for example, when the multi-lens array 800 is illuminated with a high peak power Q-switched laser source. The lateral forces can affix the lens elements 820 to one another obviating the need for an adhesive. The adhesive can produce unwanted optical effects, as well as absorb laser energy and damage the assembly 800.

As described above, commonly used microlens array manufacturing techniques may not be easily adapted for the manufacture of lens arrays having a millimeter sized pitch (e.g., millimeter sized distance between the center/centroid of adjacent lenses in the lens array) and a large N.A. (e.g. greater than 0.3, between about 0.3 and about 1). In addition to the method of manufacture described above, according to some embodiments, a multi-lens array having a millimeter sized pitch and a large N.A. can be constructed through specific single point diamond machining and glass molding techniques.

In some embodiments, a multi-lens array having the desired characteristics of long working distance and large NA may be manufactured through press molding. An exemplary glass molding contract manufacturer is Aix Tooling, GmbH of Aachen, Germany. Press molding requires a tool to be made and used as a mold. The mold is pressed against a molten substrate material (e.g., glass) in order to form a designated curvature.

In some embodiments, either the mold or multi-lens array itself is produced through single point diamond machining (SPDM) methods (e.g., micro-milling). A SPDM method well suited for production of the multi-lens arrays taught above is 4-axis SPDM. B. McCall et al. introduce 4-axis SPDM in their paper entitled “Rapid Fabrication of Miniature Lens Arrays by Four-Axis Single Point Diamond Machining,” published in Optical Society of America in 2013 and incorporated herein by reference in its entirety.

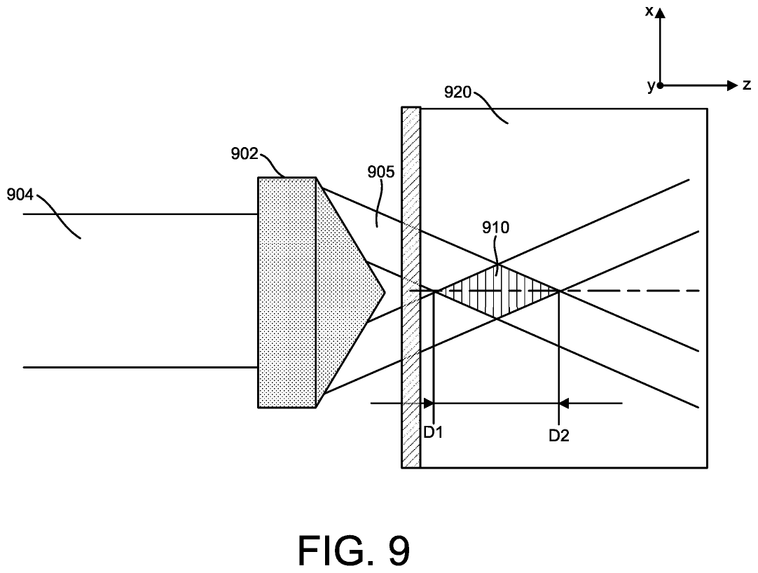

FIG. 9 is a schematic illustration of an optical element 902 configured to generate a quasi-diffraction-free beam (QDFM) having a focal region in a target tissue 920. The optical element 902 (e.g., axicon) can receive an input laser beams 904 and generate the QDFM 905. The QDFM 905 can have a focal region 910 that can extend from a first depth D1 in the target tissue to a second depth D2 in the target tissue. The optical element 902 can have a large numerical aperture (e.g., greater than 0.3, between 0.3 and 1) and a large width (e.g., from about 1 mm to about 3 mm) in the plane lateral to the direction of propagation of input laser beam 904 (e.g., in the x-y plane). The large NA can prevent undesirable interaction (e.g., generation of plasma, heating, etc.) between overlying layers of the target tissue (e.g., epidermis of a skin tissue) and QDFM 905. In some implementations, the extent of the focal region 910 along the depth of the target tissue (e.g., along z-direction) can be longer than the extent of the focal region of a beam which suffers larger diffraction (e.g., a Gaussian beam). As a result, for a given numerical aperture, a QDFM can allow for treatment along larger depths in the target tissue 920. This can obviate the need for scanning of the focal region along the depth (e.g., z-axis). As described above, the multi-lens array 730 can include one or more optical element 902.

In order to further summarize, parameters ranges associated with some embodiments are outlined in a table below:

| Parameter | Min. | Nom. | Max. |
|---|---|---|---|
| Pitch (mm) | 0.5 | 3 | 5 |
| Effective | 0.1 | 3 | 10 |
| Focal | | | |
| Length (mm) | | | |
| Working | 0.1 | 2 | 10 |
| Distance | | | |
| (mm) | | | |
| Numerical | 0.1 | 0.5 | 1 |
| Aperture | | | |
| EMR Diameter | 0.5 | 8 | 50 |
| (mm) | | | |
| No. Lens | 2 | 7 | 2000 |
| Elements | | | |
| (e.g., | | | |
| lenslets) | | | |
| No. Multi-Lens | 1 | 1 | 5 |
| Arrays | | | |

| | |
|---|---|
| Lens Array | Assembled discrete lenses, Single Point Diamond |
| Manufacturing | Machining (e.g., micro-machining, turning, and |
| Process | 4-axis machining), lithography, and molded (e.g., |
| | press molded) glass or polymer |
| Lens Array | 1 X many (line), many X many (rectangle), |
| Shape | and hexagonal |
| EMR Focusing | Scanned EMR beam focused by 1 or more lens |
| | elements at a time, and EMR beam focused by all |
| | lens elements at once. |

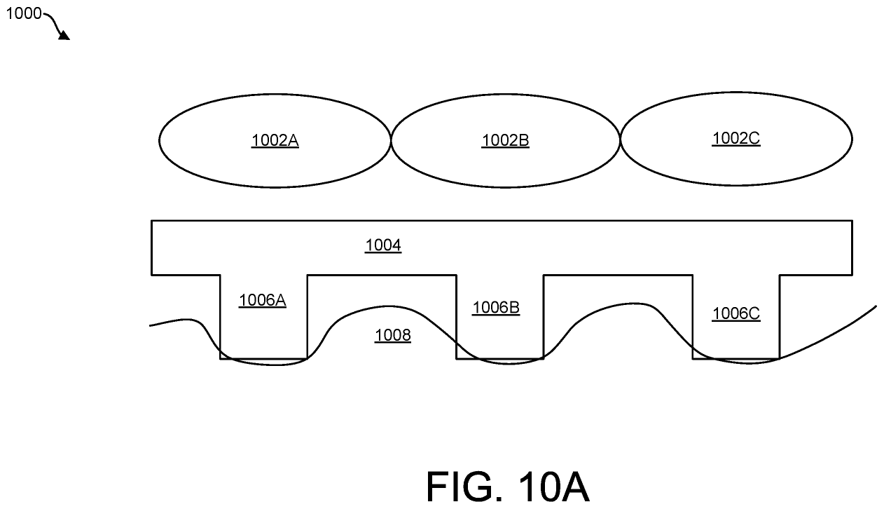

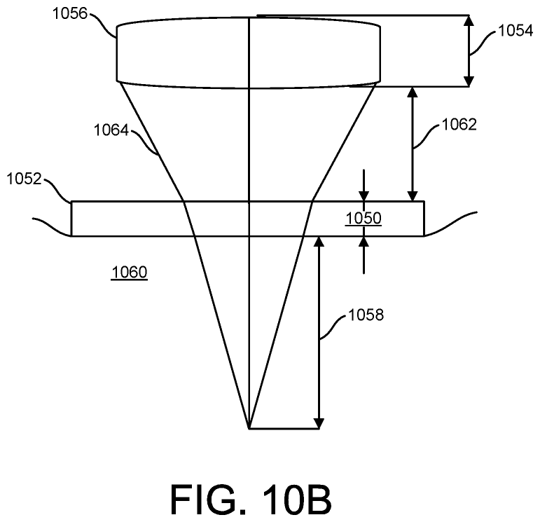

Referring to FIG. 10A, a lens array 1000 for delivering multiple beams is shown. The lens system 1000 includes a number of lens elements 1002A-1002C. A window is shown 1004. According to some embodiments, the window 1004 includes a number of protrusions 1006A-1006C which correspond to each lens element 1002A-1002C. According to some embodiments, the window 1004 contacts a surface of a skin 1008 deforming the skin surface to conform with the shape of the window 1004. For example, a flat window flattens the surface of the skin 1008 and a convex window forms an indentation in the surface of the skin 1008. According to some embodiments, the skin 1008 is deformed by the shape of the protrusions 1006A-1006C. According to some embodiments, pressure is applied by the window 1004 onto the surface of the skin 1008, and a relatively small area of the protrusions 1006A-1006C (e.g., 1 mm²) allows for greater pressure and localized compression under each element 1002A-1002C. The pressure may provide a number of functions advantageous for laser treatment including evacuating blood and other competing targets (i.e., chromophores) from the treatment region; and, condensing the thickness of the skin, thereby shrinking the optical path length to treat deeper into the skin.

Methods of treating various skin conditions, such as for cosmetic purposes, can be carried out using the systems described herein. It is understood that although such methods can be conducted by a physician, non-physicians, such as aestheticians and other suitably trained personnel may use the systems described herein to treat various skin conditions with and without the supervision of a physician.

Multi-lens arrays may be constructed from many materials including: transparent and optical polymers, sapphire, quartz, zinc-selenide, zinc-sulfide, and glass (e.g., press moldable glass). Examples of press moldable glass are manifold and include materials from Ohara (Ex. Part Nos.: L-BSL7, L-Ba135, L-Ba142, L-LAH84, and L-LAH53), Sumita (Ex. Part Nos.: K-VC89, K-PBK40, and K-CD120), and Schott (Ex. Part Nos.: P-Bk7, B270, IRG26, and Borofloat 33).

According to some methods of use, a focal region must penetrate a predetermined depth within the tissue. For some optical materials (e.g., moldable glass) and high NAs (e.g., greater than 0.3 and less than 1) a focal length of each lenslet and pitch may be approximately determined by using practical heuristics described in reference to FIG. 10B. For example, a required focal length in air may be derived using an approximation,

$${f}_{air} = \frac{{t}_{window}}{{n}_{window}} + \frac{{t}_{substrate}}{{n}_{substrate}} + \frac{{t}_{MAX tissue depth}}{{n}_{MAX tissue depth}} + \frac{{t}_{air}}{{n}_{air}}$$

where, fₐᵢᵣ is focal length of the lenslet in air; t_{window} 1050 and n_{window} are thickness of a window 1052 and index of refraction of the window 1052 respectively; t substrate 1054 and n_{substrate} are thickness of an optical substrate 1056 (e.g., focus optic) and index of refraction of the substrate 1056 respectively; t_{MAX tissue depth} and n_{MAX tissue depth} are thickness of a max desired focal depth 1058 in the tissue 1060 and index of refraction of the tissue 1060 respectively; and, tₐᵢᵣ 1062 and nₐᵢᵣ are the thickness 1062 of an air gap 1064 and index of refraction of the air gap 1064 (e.g., 1), respectively;

According to some embodiments, a pitch of a multi-lens array is between one-half and four-times a focal length of a lenslet of the multi-lens array (in air). For example, this relationship is:

$$\frac{{f}_{air}}{2} \leq p \leq 4 {f}_{air}$$

where, fₐᵢᵣ is a focal length of a lenslet of the multi-lens array in air; and, p is a pitch between lenslets in the multi-lens array.

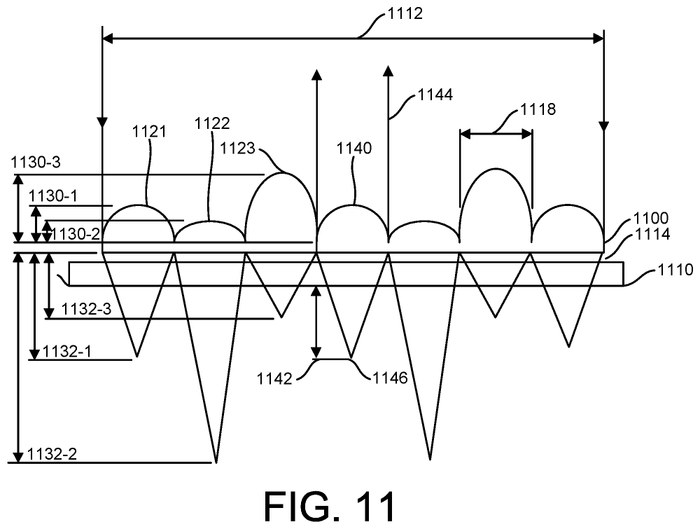

In some embodiments, a multi-lens array 1100 comprises lenslets having focal lengths of varied lengths. Referring to FIG. 11, shows a multi-lens array 1100 being used to irradiate a tissue 1110. An EMR beam 1112 is projected incident the multi-lens array 1100. A transmissive window 1114 is placed in contact with an outer surface of the tissue 1110. The window 1114 typically has a thickness. An air gap 1116 exists between the window 1114 and the multi-lens array 1100. The air gap 1116 typically has a thickness. The multi-lens array 1100 includes a number (e.g., 7) of lenslets each separated by a pitch 1118. The pitch 1118 may be a maximum diameter of a lenslet or a spacing between optical axes of adjacent lenslets. 3 lenslets, a first example lenslet 1121, a second example lenslet 1122, and a third example lenslet 1123 on the left of the array 1100 are explained in detail, as an example. Each, lenslet includes a curvature, a sag, and a focal length. The first example lenslet 1121 has a first curvature, a first sag 1130-1, and a first focal length 1132-2. The second example lenslet 1122 has a second curvature, a second sag 1130-2 less than the first sag 1130-1, and a second focal length 1132-2 greater than the first focal length 1132-1. The third example lenslet 1123 has a third curvature, a third sag 1130-3 greater than the first sag 1130-1, and a third focal length 1132-3 shorter than the first focal length 1132-1.

According to some embodiments, a reference lenslet 1140 is used to determine a reference focal depth 1142. Light reflecting 1144 from a referencing focal region 1146 may be collimated by the referencing lenslet 1140. The collimated light 1144 may be used to determine the reference focal depth 1142. Methods and systems for performing referencing in this manner are described in detail in U.S. Provisional Patent Application No. 62/688,940 entitled “Radiation Detection for a Treatment Device” by J. Bhawalkar et al., and is incorporated herein by reference.

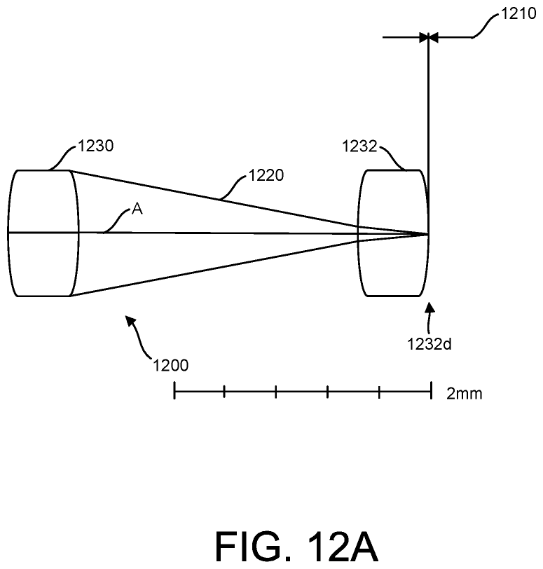

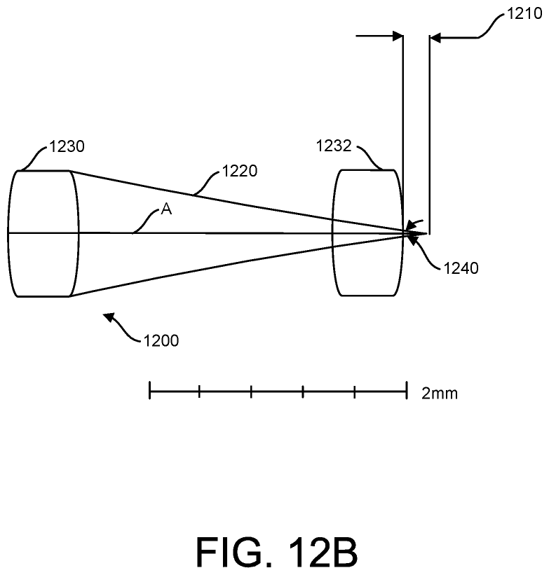

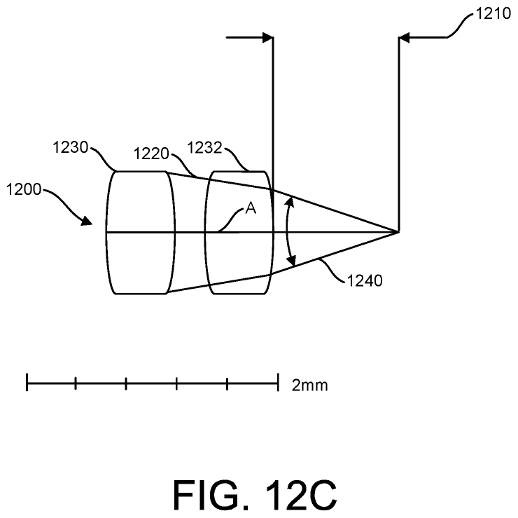

Referring to FIGS. 12A-12C, in some embodiments, a variable assembly 1200 may be used to vary back focal distance 1210. For example, 2 multi-lens assemblies may be used together. In some embodiments, using 2 or more multi-lens assemblies reduces a maximum sag distance required. This is because, all of a total curvature required to focus an EMR beam can be distributed over multiple surfaces. For Example, 2 multi-lens assemblies have 4 surfaces, which may have a curvature. Decreasing the sag distance of an optic typically makes the optic easier to manufacture.

FIGS. 12A-12C show a view of a single EMB beamlet 1220 being focused by a pair of single lenslets 1230 and 1232. FIG. 12A illustrates the first lenslet 1230 at a maximum spacing from the second lenslet 1232 along axis A. The back focal distance 1210 in this configuration is 0. Said another way, the focal region is located at a distal surface 1232d of the second lenslet 1232.

FIG. 12B illustrates the first lenslet 1230 at a medium spacing from the second lenslet 1232 along axis A. The back focal distance 1210 in this configuration has increased from 0 to a medium focal distance (e.g., 0.5 mm). Also, it can be seen in FIG. 12B that the EMR beamlet is being focused at a convergence angle 1240.

FIG. 12C illustrates the first lenslet 1230 at a minimum spacing from the second lenslet 1232. As a result, the back focal distance 1210 in this configuration is greatest (e.g., 1 mm). Likewise, the convergence angle 1240 is shown to be greatest with a minimum spacing between the first lenslet 1230 and the second lenslet 1232. Numerical aperture (NA) can be used as a measure of the convergence angle 1240. In some embodiments, the numerical aperture of the focusing EMR beamlet(s) changes by a factor of 2 between the maximum lenslet spacing and the minimum lenslet spacing.

Variable focus using multiple multi-lens arrays may be approximated (using the paraxial assumption) with a thin-lens equation for compound lenses. The thin-lens equation for compound lenses allows an effective focal length of a lens assembly to be calculated from focal lengths of 2 (or more) optics and a spacing between principal planes of those optics.

$$\frac{1}{f} = \frac{1}{{f}_{1}} + \frac{1}{{f}_{2}} - \frac{d}{{f}_{1} {f}_{2}}$$

where, f is the effective focal length of the lens assembly; f₁ is the focal length of a first optic; f₂ is the focal length of a second optic; and, d is a distance between the first optic and the second optic (e.g., along axis A).

### Additional Embodiments

In some embodiments, the repetition rate of the input laser beam can be faster than the decay rate of the plasma in the target tissue/target material. This can allow for continuous (e.g., temporally continuous, spatially continuous, etc.) generation of plasma. The area of the treatment region/target region (e.g., region in which plasma is generated) can be controlled by changing the repetition rate of the laser beam.

One skilled in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

Approximating language, as used herein throughout the specification and claims, may be applied to modify any quantitative representation that could permissibly vary without resulting in a change in the basic function to which it is related. “Approximately,” “substantially, or “about” can include numbers that fall within a range of 1%, or in some embodiments within a range of 5% of a number, or in some embodiments within a range of 10% of a number in either direction (greater than or less than the number) unless otherwise stated or otherwise evident from the context (except where such number would impermissibly exceed 100% of a possible value). Accordingly, a value modified by a term or terms, such as “about,” approximately, or “substantially,” is not to be limited to the precise value specified. In at least some instances, the approximating language may correspond to the precision of an instrument for measuring the value. Here and throughout the specification and claims, range limitations may be combined and/or interchanged, such ranges are identified and include all the sub-ranges contained therein unless context or language indicates otherwise.

The articles “a” and “an” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure also includes embodiments in which more than one, or all of the group members are present in, employed in, or otherwise relevant to a given product or process. Furthermore, it is to be understood that the disclosed embodiments provide all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. It is contemplated that all embodiments described herein are applicable to all different aspects of the disclosed embodiments where appropriate. It is also contemplated that any of the embodiments or aspects can be freely combined with one or more other such embodiments or aspects whenever appropriate. Where elements are presented as lists, e.g., in Markush group or similar format, it is to be understood that each subgroup of the elements is also disclosed, and any element(s) can be removed from the group. It should be understood that, in general, where the disclosed embodiments, or aspects of the disclosed embodiments, is/are referred to as comprising particular elements, features, etc., certain embodiments of the disclosure or aspects of the disclosure consist, or consist essentially of, such elements, features, etc. For purposes of simplicity those embodiments have not in every case been specifically set forth in so many words herein. It should also be understood that any embodiment or aspect of the disclosure can be explicitly excluded from the claims, regardless of whether the specific exclusion is recited in the specification. For example, any one or more active agents, additives, ingredients, optional agents, types of organism, disorders, subjects, or combinations thereof, can be excluded.

Where ranges are given herein, embodiments of the disclosure include embodiments in which the endpoints are included, embodiments in which both endpoints are excluded, and embodiments in which one endpoint is included and the other is excluded. It should be assumed that both endpoints are included unless indicated otherwise. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise. It is also understood that where a series of numerical values is stated herein, the disclosure includes embodiments that relate analogously to any intervening value or range defined by any two values in the series, and that the lowest value may be taken as a minimum and the greatest value may be taken as a maximum. Numerical values, as used herein, include values expressed as percentages.

Any embodiment in which a numerical value is prefaced by “about” or “approximately” includes an embodiment in which the exact value is recited. For any embodiment of the disclosure in which a numerical value is not prefaced by “about” or “approximately”, the disclosure includes an embodiment in which the value is prefaced by “about” or “approximately.” “Approximately” or “about” can include numbers that fall within a range of 1% or in some embodiments within a range of 5% of a number or in some embodiments within a range of 10% of a number in either direction (greater than or less than the number) unless otherwise stated or otherwise evident from the context (except where such number would impermissibly exceed 100% of a possible value).

It should be understood that, unless clearly indicated to the contrary, in any methods claimed herein that include more than one act, the order of the acts of the method is not necessarily limited to the order in which the acts of the method are recited, but the disclosure includes embodiments in which the order is so limited. It should also be understood that unless otherwise indicated or evident from the context, any product or composition described herein may be considered “isolated.”

As used herein the term “comprising” or “comprises” is used in reference to compositions, methods, and respective component(s) thereof, that are essential to the disclosed embodiments, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the disclosure.

The term “consisting of” refers to compositions, methods, and respective components thereof as described herein, which are exclusive of any element not recited in that description of the embodiment.

Although a few variations have been described in detail above, other modifications or additions are possible.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and sub-combinations of the disclosed features and/or combinations and sub-combinations of several further features disclosed above. In addition, the logic flows depicted in the accompanying figures and/or described herein do not necessarily require the particular order shown, or sequential order, to achieve desirable results. Other implementations may be within the scope of the following claims.

What is claimed is:

1. An optical system comprising:
an array of optical elements configured to receive a primary laser beam and generate a plurality of sub-beams, the array of optical elements comprising a plurality of optical elements configured to simultaneously focus the plurality of sub-beams to a plurality of focal regions in a target tissue,
wherein a pitch of the array of optical elements ranges from about 1 mm to about 3 mm,
wherein a numerical aperture of one or more optical elements of the plurality of optical element ranges from about 0.3 to about 1, and
wherein a first sub-beam of the plurality sub-beams is configured to generate plasma in a first focal region of the plurality of focal regions.

2. The optical system of claim 1, wherein the plurality of optical elements include a plurality of truncated lenses.

3. The optical system of claim 1, wherein a width of the plurality of optical elements ranges from about 1 mm to about 3 mm.

4. The optical system of claim 2, wherein the plurality of truncated lenses are arranged in at least one of a hexagonal array and a rectangular array.

5. The optical system of claim 1, further comprising a window configured to contact a tissue and transmit the plurality of sub-beams.

6. The optical system of claim 1, wherein the first sub-beam is configured to generate plasma thermionically.

7. The optical system of claim 1, wherein the first sub-beam is configured to generate plasma optically.

8. The optical system of claim 1, wherein the plurality of optical elements are held together by a holder configured to apply a lateral force on one or more optical elements of the plurality of optical elements.

9. A method comprising:
receiving, by an array of optical elements comprising a plurality of optical elements, a primary laser beam;
generating, by the plurality of optical elements, a plurality of sub-beams focused at a plurality of focal regions in a target tissue;
wherein a pitch of the array of optical elements ranges from about 1 mm to about 3 mm;
wherein a numerical aperture of one or more optical elements of the plurality of optical element ranges from about 0.3 to about 1; and
wherein a first sub-beam of the plurality sub-beams is configured to generate plasma in a first focal region of the plurality of focal regions.

10. The method of claim 9, wherein the plurality of optical elements include a plurality of truncated lenses.

11. The method of claim 9, wherein a width of the plurality of optical elements ranges from about 1 mm to about 3 mm.

12. The method of claim 10, wherein the plurality of truncated lenses are arranged in at least one of a hexagonal array and a rectangular array.

13. The method of claim 9, further comprising:
contacting, using a window, a tissue; and
transmitting the plurality of sub-beams through the window.

14. The method of claim 9, wherein the first sub-beam is configured to generate plasma thermionically.

15. The method of claim 9, wherein the first sub-beam is configured to generate plasma optically.

16. The method of claim 9, wherein the plurality of optical elements are held together by a holder configured to apply a lateral force on one or more optical elements of the plurality of optical elements.

17. A tissue treatment system comprising:
a laser system configured to emit a primary laser beam;
an array of optical elements configured to receive the primary laser beam and generate a plurality of sub-beams, the array of optical elements comprising a plurality of optical elements configured to simultaneously focus the plurality of sub-beams to a plurality of focal regions in a target tissue;
wherein a pitch of the array of optical elements ranges from about 1 mm to about 3 mm;
wherein a numerical aperture of one or more optical elements of the plurality of optical element ranges from about 0.3 to about 1; and
wherein a first sub-beam of the plurality sub-beams is configured to generate plasma in a first focal region of the plurality of focal regions.
